(19) United States
(12) Patent Application Publication
(10) Pub. No.: US 2023/0051852 A1
(43) Pub. Date: Feb. 16, 2023

(54) CONNECTOR ASSEMBLY FOR CONNECTING A ROBOTIC ARM WITH A SURGICAL END EFFECTOR

(71) Applicant: NuVasive, Inc., San Diego, CA (US)

(72) Inventors: Todd Baxendale, Broomfield, CO (US); Cara Coad, Longmont, CO (US); Thomas Sweeney, San Diego, CA (US)

(21) Appl. No.: 17/400,888

(22) Filed: Aug. 12, 2021

(51) Int. Cl. A61B 34/30; A61B 46/10; A61F 2/46

(52) U.S. Cl. CPC A61B 34/30; A61B 46/10; A61F 2/4611; A61B 2017/00486

(57) ABSTRACT

Medical devices and connector assemblies for connecting medical devices are disclosed. An example connector assembly for connecting a robotic arm with a medical end effector may include a plate configured to be coupled to a robotic arm. The plate may have a connection region that includes a flange defining a circumferential groove. The connector assembly may also include an attachment assembly having an attachment region configured to be detachably secured to the connection region with a securing distance of less than 12 millimeters. The attachment region may include one or more engagement members that are configured to shift between an unsecured position and a secured position where the engagement members are secured to the connection region. An actuator may be coupled to the attachment assembly for shifting the one or more engagement members between the unsecured position and the secured position.

### TECHNICAL FIELD

The present disclosure pertains to surgical medical devices. More particularly, the present disclosure pertains to connectors for connecting a robotic arm with a surgical end effector.

### BACKGROUND

There are a wide variety of surgical medical devices. Some of these devices include robotic arms, surgical end effectors, and the like. Of the known surgical medical devices, each has certain advantages and disadvantages. There is an ongoing need to provide alternative surgical medical devices.

### BRIEF SUMMARY

This disclosure provides design, material, manufacturing method, and use alternatives for medical devices. A connector assembly for connecting a robotic arm with a medical end effector is disclosed. The connector assembly comprises: a plate configured to be coupled to a robotic arm, the plate having a connection region that includes a flange defining a circumferential groove; an attachment assembly having an attachment region configured to be detachably secured to the connection region with a securing distance of less than 12 millimeters; wherein the attachment region includes one or more engagement members that are configured to shift between an unsecured position and a secured position where the engagement members are secured to the connection region; and an actuator coupled to the attachment assembly for shifting the one or more engagement members between the unsecured position and the secured position.

Alternatively or additionally to any of the embodiments in this section, the plate includes an alignment opening, the alignment opening being configured to allow a sterile barrier adapter to be aligned with the plate.

Alternatively or additionally to any of the embodiments in this section, further comprising: a region of a flexible sterile barrier wedged between the connection region and the attachment assembly, wherein the region lacks an adapter.

Alternatively or additionally to any of the embodiments in this section, the one or more engagement members includes one or more wedge tabs configured to engage the circumferential groove.

Alternatively or additionally to any of the embodiments in this section, the actuator includes a latch.

Alternatively or additionally to any of the embodiments in this section, the plate includes an alignment region.

Alternatively or additionally to any of the embodiments in this section, the end effector has an alignment member configured to engage the alignment region of the plate.

Alternatively or additionally to any of the embodiments in this section, the securing distance is defined by the depth of the puck.

Alternatively or additionally to any of the embodiments in this section, the connection region comprises one or more magnetic field sensors configured to detect one or more magnets of the attachment assembly.

A medical assembly is disclosed. The medical assembly comprises: a plate configured to be coupled to a robotic arm, the plate having a connection puck and one or more locating regions; an end effector having an attachment region configured to receive the connection puck and be detachably secured to the connection puck, wherein the attachment region includes: one or more locating members configured to engage the one or more locating regions of the connection puck so that a sterile barrier can be disposed between the attachment region and the connection puck without requiring a sterile barrier adapter; one or more engagement members that are configured to shift between an unsecured position and a secured position where the engagement members are secured to the connection puck; and an actuator coupled to the end effector configured to shift the one or more engagement members between the first position and the secured position.

Alternatively or additionally to any of the embodiments in this section, the connection puck has a circumferential groove formed therein.

Alternatively or additionally to any of the embodiments in this section, the one or more engagement members includes one or more wedge tabs configured to engage the circumferential groove.

Alternatively or additionally to any of the embodiments in this section, the actuator includes a latch.

Alternatively or additionally to any of the embodiments in this section, the attachment region is configured to be detachably secured to the connection puck with a securing distance of 12 millimeters or less.

Alternatively or additionally to any of the embodiments in this section, the securing distance has a length of 10-12 millimeters.

Alternatively or additionally to any of the embodiments in this section, the end effector is a surgical tool guide.

A method is disclosed. The method comprises: disposing a region of a sterile barrier lacking an adapter over a connection puck of a plate coupled to a robotic arm, wherein the connection puck includes a plurality of locating regions; engaging an attachment region of a medical end effector with the connection puck, wherein the engaging includes: inserting the connection puck into the attachment region, engaging a plurality of locating members of the attachment region with the plurality of locating regions of the connection puck, and sandwiching the sterile barrier between the attachment region and the connection puck; and actuating an actuator of the attachment region to shift one or more engagement members from an unsecured position to the secured position.

Alternatively or additionally to any of the embodiments in this section, actuating the actuator causes one or more wedge tabs of the one or more engagement members to move into a circumferential groove of the connection puck.

Alternatively or additionally to any of the embodiments in this section, further comprising: actuating the actuator of the attachment region to shift the one or more engagement members from the secured position and to an unsecured position; freeing the end effector from the connection puck by axially withdrawing the end effector from the connection puck no more than 12 millimeters; and moving the end effector laterally after freeing the end effector.

Alternatively or additionally to any of the embodiments in this section, further comprising: after actuating the actuator, performing a spinal fusion procedure with the robot arm, the end effector, or both.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is and exploded view of an example connector assembly.

FIG. 2 is a perspective view of a portion of an example connector assembly.

FIG. 3 is a perspective view of a portion of an example connector assembly.

FIG. 4 is a perspective view of a portion of an example connector assembly.

FIG. 5 is a partial cross-sectional view of an example connector assembly.

FIG. 6 is a perspective view of a portion of an example connector assembly.

FIG. 7 is a partial cross-sectional view of an example connector assembly.

FIG. 8 is and exploded view of an example connector assembly.

FIG. 9 is a perspective view of a portion of an example connector assembly.

FIG. 10 is a perspective view of a portion of an example connector assembly.

FIG. 11 is a perspective view of a portion of an example connector assembly.

FIG. 12 is a perspective view of a portion of an example connector assembly.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

A number of medical procedures, such as spinal surgeries, utilize robotic structures and/or tools that can be manipulated by robotic arms. Such robotic structures may include robotic arms with a connector that allows for tools (e.g., tools suitable for a given intervention) to be attached to the robotic arm. It can be appreciated that a medical procedure may need to utilize a number of different tools in conjunction with the robotic arm. Doing so may necessitate swapping out of various tools at different times during the procedure. Disclosed herein are connectors that can be used with various robotic structures. Such connectors may allow for relatively quick and efficient connection/disconnection of tools and/or provide other benefits as disclosed herein.

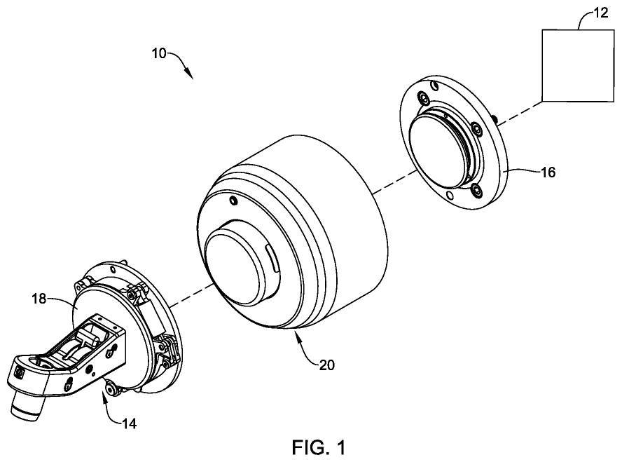

FIG. 1 depicts a connector assembly 10, which generally can be used for connecting or otherwise securing a robotic arm 12 (shown schematically in FIG. 1) and a medical end effector 14 (e.g., a tool, a spinal tool, a tool holder, an instrument holder, combinations thereof, and/or the like). The connector assembly 10 may include a plate 16 that can be attached to the robotic arm 12, for example using one or more fasteners. The connector assembly 10 may also include an attachment assembly 18 that may be integral with or otherwise secured to the end effector 14. Securing the end effector 14 to the robotic arm 12 may be accomplished by securing the attachment assembly 18 to the plate 16. In some instances, a sterile barrier adapter 20 can be disposed between the plate 16 and the attachment assembly 18. The sterile barrier adapter 20 may help to facilitate separation of objects inside and outside of the sterile field.

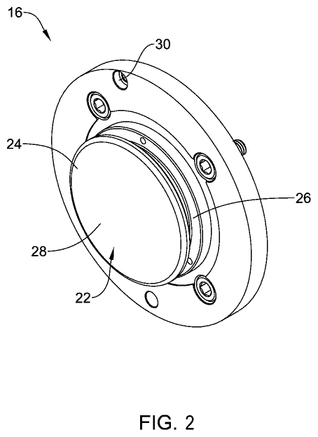

FIG. 2 depicts the plate 16. The plate 16 may include a connection region 22. In some instances, the connection region 22 may include a flange or puck 24 and a circumferential groove 26. In some instances, the groove 26 is formed at an angle that is substantially normal to the longitudinal axis of the connection region 22. In other words, the groove 26 is formed between walls that are essentially perpendicular to the longitudinal axis of the connection region 22. In other instances, the groove 26 may be beveled and/or otherwise formed at a slight angle. For example, the groove 26 may be angled about 5-20 degrees, or about 8-15 degrees, or about 10 degrees (e.g., angled from perpendicular in the direction toward the face or front surface 28 of the puck 24). The angle may help to more easily guide the puck 24 into the groove 26.

The use of the connection region 22 that includes a puck 24 and groove 26 may be desirable for a number of reasons. For example, the puck 24 may be configured so that the groove 26 is relatively close to the front surface 28 of the puck 24. Because of this, a relatively short “attachment distance” may be formed/defined by the puck 24 and groove 26. For the purposes of this disclosure, the attachment distance may be understood as the amount of distance that the plate 16 and the attachment assembly 18 need to be moved in order to attach and/or detach the end effector 14 and/or the robotic arm 12. Stated another way, the attachment distance may be understood as the amount of distance that the plate 16 and the attachment assembly 18 need to be separated or spaced apart from one another during detachment so that they can be moved apart without contacting/interacting with one another. For example, during a procedure in which it is desired for the end effector 14 to be swapped out with another end effector, it would be necessary to detach the end effector 14 from the robotic arm 12 (e.g., detach the attachment assembly 18 from the plate 16) and then move the end effector 14 away from the robotic arm 12 so that it can be replaced by another end effector. In this example, the attachment assembly 12 would need to be shifted away from the plate 16 by a distance greater than or equal to the attachment distance. The length of the attachment region may correspond to or otherwise be defined by the distance between the front surface 28 of the puck 24 and the groove 26. By having a relatively short attachment distance, the distance that is needed to move the end effector 14 away from the plate 16 is kept relatively short, making it easier to swap out tools quickly and efficiently while reducing the likelihood of disrupting the position of the robotic arm 12 (e.g., which may be important when performing a procedure where positional precision is desired). This may also allow for quick release of the end effector 14 from the robotic arm 12 if an urgent need or emergency arises. In some instances, the attachment distance may be about 16 millimeters or less, or about 14 millimeters or less, or about 12 millimeters or less, or about 10 millimeters or less, or about 2-16 millimeters, or about 8-14 millimeters, or about 10-12 millimeters.

In addition to allowing for connection between the plate 16/robotic arm 12 and the attachment assembly 18/end effector 14, the puck 24 and groove 26 on the plate 16 may allow for a connection/securement between the plate 16 and the end effector 14 at a number of different locations about the circumference of the puck 24/groove 26. Because of this, the connection between the end effector 14 and the plate 16 may have an increased stability, which may also help maintain positional precision. For example, in tests to measure the stability of the connection, the connection was shown to withstand 10 pounds of force with minimal deflection (e.g., about 0.10 millimeters or less of deflection). Such tests may include using a fixture that allows for the application of force at a location corresponding to where the theoretical tip center point of the longest pedicle screw contemplated for use would be (e.g., while engaged with a screw driver coupled to the end effector 14).

The plate 16 may also include one or more alignment or locating openings 30. In some instances, the alignment opening(s) 30 may aid in the placement/alignment of a sterile barrier adapter 20 between the plate 16/robotic arm 12 and the attachment assembly 18/end effector 14.

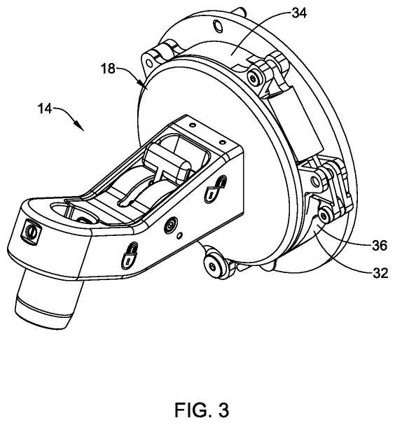

FIG. 3 depicts the end effector 14. As indicated above, the end effector 14 may take the form of a tool, a surgical tool, a tool holder, an instrument holder, combinations thereof, and the like. The depiction of the end effector 14 is the figures is not intended to be limiting as various end effectors 14 can be utilized. As shown in FIG. 3, the end effector 14 may include the attachment assembly 18 with an attachment region 32. In some instances, the attachment assembly 18 is an integral part of the end effector 14. In other instances, the attachment assembly 18 is a separate structure that can be secured to the end effector 14. In some of these and in other instances, the attachment assembly 18, along with the plate 16, may be considered to be part of the connector assembly 10 (e.g., and the robotic arm 12, the end effector 14, or both may or may not be considered to be part of the connector assembly 10). The attachment region 32 may include one or more engagement members 34. The engagement members 34 may be used to help secure the attachment assembly 18 to the connection region 22 of the plate 16. In some instances, the engagement members 34 may take the form of wedge tabs that can be disposed within the groove 26 of the connection region 22. A suitable number of engagement members 34 may be utilized. For example, one, two, three, four, five, six, or more engagement members 34 may be utilized. The use of a plurality (e.g., two or more) engagement members 34 may be desirable and may allow for greater precision when swapping end effectors 14 and/or provide multiple points of contact between the attachment assembly 18 and the connection region 22.

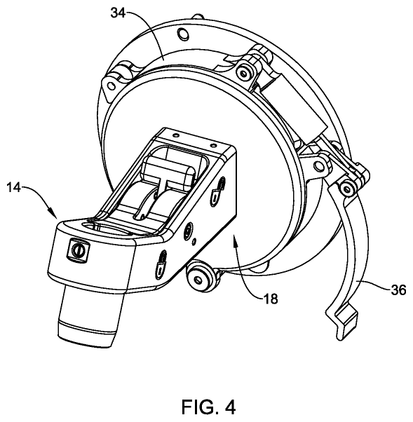

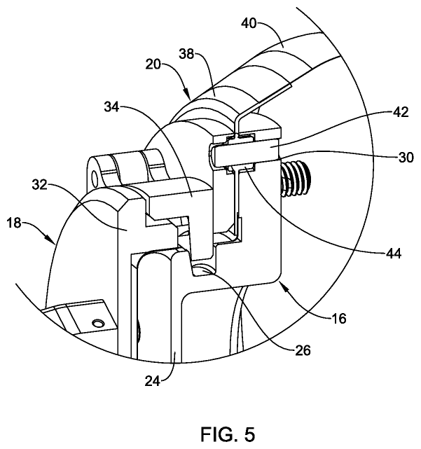

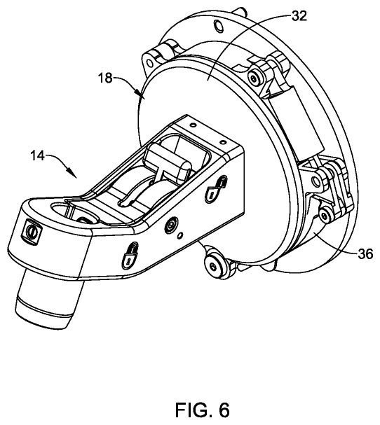

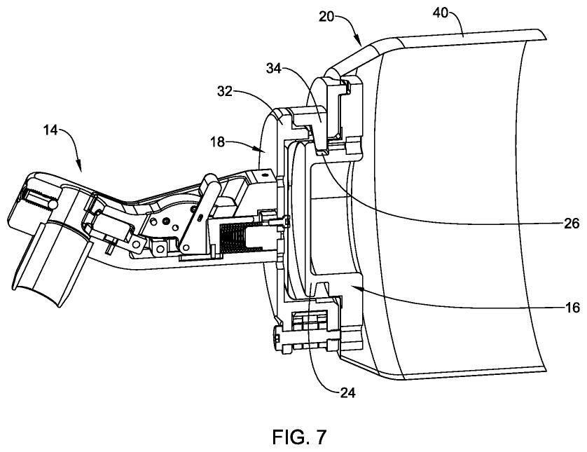

The attachment assembly 18 may also include an actuator 36. The actuator 36 may be used to shift the engagement members 34 between unsecured position (e.g., as shown in FIGS. 4-5) and a secured position (e.g., as shown in FIGS. 6-7) where the engagement members 34 are secured to the connection region 22 of the plate 16. In some instances, the actuator 36 may take the form of a latch (e.g., an over-center latch) or otherwise include a handle/graspable region that can be actuated by a user. For example, as shown in FIGS. 4-5, the actuator 36 may be in a position that could be described as “open” or “unsecured”. The actuator 36 may include a spring that biases the actuator 36 toward the “unsecured” position. When in this position, the handle or graspable portion of the actuator 36 is pulled away from the attachment region 32. This shifts the engagement members 34 radially outward relative to the attachment region 32 so that the attachment region 32 can be moved toward or away from the puck 24. Conversely, as shown in FIGS. 6-7, the actuator 36 may be shifted to a position that could be described as “closed” or “secured”. When in this position, the handle or graspable portion of the actuator 36 is urged closer to the attachment region 32. This shifts the engagement members 34 radially inward relative to the attachment region 32 so that the engagement members 34 fit into the groove 26 and so that the attachment region 32 is secured to the puck 24.

Also shown in FIGS. 5 and 7 is the sterile barrier adapter 20. The sterile barrier adapter 20 may include an adapter ring 38 and a sterile barrier 40. In some instances, an alignment pin 42 may extend through the alignment opening 30 and an opening 44 in the adapter ring 38.

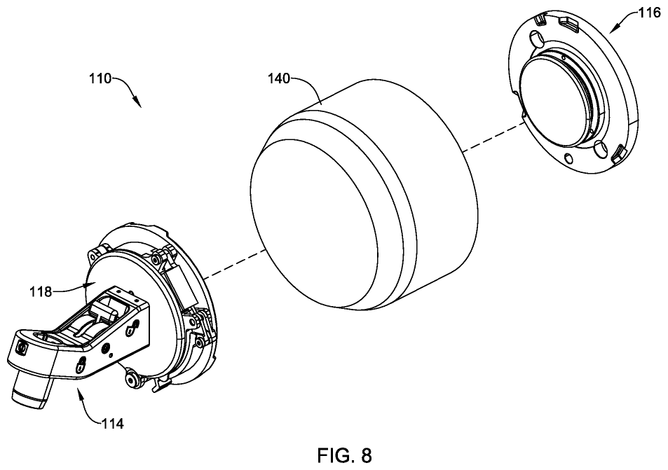

FIG. 8 illustrates another example connector assembly 110 that may be similar in form and function to other connector assemblies disclosed herein. The connector assembly 110 may include a plate 116 (e.g., which may be securable to a robotic arm such as those disclosed herein), an attachment assembly 118 (e.g., which may be securable to or integral with an end effector 114), and a sterile barrier 140 (e.g., which lacks the sterile barrier adapter discussed above).

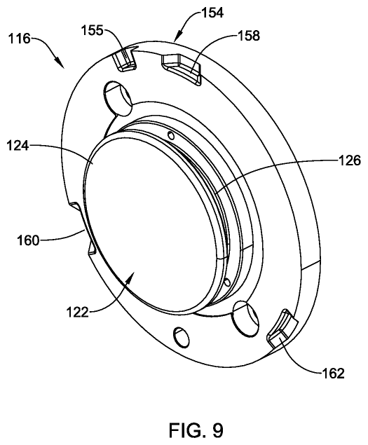

FIG. 9 illustrates the plate 116. Here it can be seen that the plate 116 may include a connection region 122. In some instances, the connection region 122 may include a flange or puck 124 and a circumferential groove 126. These structures may function similarly to those in the plate 16 described above.

The plate 116 may include an alignment or locating region 154. The alignment region 154 may include one or more cutouts or grooves such as cutout 155 and cutout 158. One or more additional cutouts such as cutouts 160, 162 may also be located about the plate 116. In general, the cutouts 155, 158 (and/or cutouts 160, 162) may help align the connection region 122 of the plate 116 with the attachment assembly 118. More particularly, the shape the cutouts 155, 158, 160, 162 may correspond to and/or be configured to mate with corresponding shapes or projections (e.g., as described below) on the attachment assembly 118. In some instances, the cutouts 155, 158, 160, 162 are distributed about the connection region 122 in a pattern that permits the attachment assembly 118 from inadvertently being misaligned with the connection region 122 of the plate 116. For example, the cutouts 155, 158, 160, 162 may be distributed asymmetrically or in a manner that allows the attachment assembly 118 to engage the connection region 122 in a singular orientation. In some instances, cutouts 158, 162, 160 may be distributed about the plate 116 and the cutout 155 may function to prevent inadvertent misalignment (e.g., because the presence of the cutout 155 may make it impossible for the attachment assembly 118 to mate/engage with the plate 116 in any other orientation than the desired orientation). The locations of the cutouts 155, 158, 160, 162 may form kinematic constraints that constrain the assembly in all degrees of freedom and allow the attachment assembly 118 to be removed and replaced with a relatively tight spherical accuracy (e.g., within about 0.2 mm or less, or about 0.05 mm or less, or about 0.02 mm or less). In other words, the cutouts 155, 158, 160, 162 may help to form a kinematic coupling between the plate 116 and the attachment assembly 118 with a high degree of positional repeatability while constraining the coupling in all degrees of freedom. In addition, the shape of the cutouts 155, 158, 160, 162 may vary from one another. For example, in some instances, the shape or size of one or more of the cutouts 155, 158, 160, 162 differs from one or more of the other cutouts 155, 158, 160, 162. This also helps to aid in aligning the attachment assembly 118 with the connection region 122.

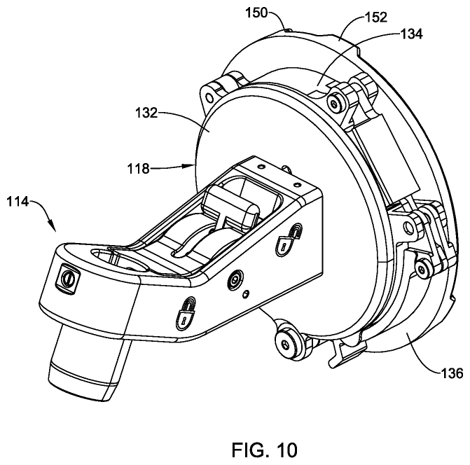

FIG. 10 depicts the end effector 114 and the attachment assembly 118. The attachment assembly 118 may include an attachment region 132. The attachment region 132 may include one or more engagement members 134 and an actuation member 136, which may be similar those disclosed herein. The engagement members 134 may be used to help secure the attachment assembly 118 to the plate 116 (e.g., the connection region 122 of the plate 116) in a manner similar to what is described herein. In some instances, the engagement members 134 may take the form of wedge tabs. The combination of the attachment assembly 118 and the connection region 122 may define a relatively short attachment distance, similar to that described herein. For example, the attachment distance may be about 16 millimeters or less, or about 14 millimeters or less, or about 12 millimeters or less, or about 10 millimeters or less, or about 2-16 millimeters, or about 8-14 millimeters, or about 10-12 millimeters.

The attachment assembly 118 may include one or more alignment members such as alignment members 150, 152. In this example, the alignment members 150, 152 are designed to engage and/or mate with the cutouts 155, 158, respectively. This allows the attachment assembly 118 to be brought into contact with the connection region 122 in a desired orientation. Moreover, given that the orientation can be controlled with a desired level of precision, additional orienting features (e.g., such as those for aligning a sterile barrier adapter) are not required. Indeed, the controlled orientation of the attachment assembly 118 relative to the connection region 122 allows for a sterile barrier or drape to be draped over the connection region 122 without needing an adapter. This may aid in the maintaining sterility as well as obviate the need for a particular sterile barrier adapter with a particular orienting feature, which may simplify the process of connecting/disconnecting the attachment assembly 118 to the connection region 122.

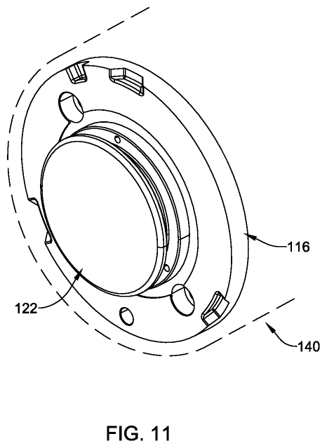

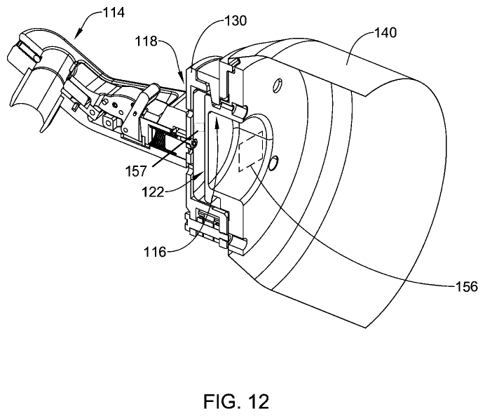

FIGS. 11-12 depict the connection of the attachment assembly 118 with the connection region 122 of the plate 116. In FIG. 11, a sterile barrier 140 is shown overlying the connection region 122 (e.g., without an adapter). With the barrier 140 overlying the connection region 122, the attachment assembly 118 can brought into engagement with the connection region 122. This may include the alignment members 150, 152 engaged and/or mated with the cutouts 155, 158, respectively. The sterile barrier 140 can be sandwiched in between the connection region 122 and the attachment assembly 118.

In some instances, the plate 116 (e.g., the connection region 122) may include one or more sensors, such as one or more magnetic field sensors 156 (e.g., Hall effect sensors). The sensors 156 may be configured to detect one or more magnets 157 (and/or a magnetic region) of the attachment assembly 118 and/or detect the position of the magnets. This may allow for connection between the connection region 122 and the attachment assembly 118 to be detected/sensed.

In addition, the presence or absence of one or more magnets in particular locations can be used to convey information regarding the identity of the end effector 114. For instance, there may be n magnet sensors 156 grouped in the connection region 122 to produce an output usable to determine the identity of the end effector based on the presence or absence of magnets in predetermined locations at the end effector 114. The output of the n magnet sensors 156 can represent the binary form of a number 0 to n²−1 (e.g., with a magnet sensor 156 not detecting a magnet indicating 0 and a sensor detecting the presence of a magnet indicating 1). The resulting number can express an identity of the end effector 114 to the robot.

Further, one or more magnets can be used to determine a status of a component of the end effector 114. For example, the illustrated end effector 114 includes a lever for locking a position of a tool inserted into the end effector 114. When the lock is engaged, a magnet in the end effector 114 can be moved close enough to the magnet sensor 156 to be detected, but the magnet is too far away when the lock is disengaged. In such a manner, the lock/unlock status of the end effector 114 can be determined.

The information conveyed by the magnets and sensed by the magnet sensors 156 can permit different capabilities of the robot can be activated or deactivated depending on the output of the magnet sensors. The use of magnets can be advantageous by not requiring an electrical connection through the sterile barrier (which can increase complexity of the system) and by not requiring active electronics in the end effector 114, which may be damaged during a sterilization process.

In some instances, power may be passed through the plate 116 (e.g., the connection region 122), for example in order to power devices such as LEDs and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The detailed description should be read with reference to the drawings in which similar elements in different drawings are numbered the same. The drawings, which are not necessarily to scale, depict illustrative embodiments and are not intended to limit the scope of the invention.

What is claimed is:

1. A connector assembly for connecting a robotic arm with a medical end effector, the connector assembly comprising:
a plate configured to be coupled to a robotic arm, the plate having a connection region that includes a flange defining a circumferential groove;
an attachment assembly having an attachment region configured to be detachably secured to the connection region with a securing distance of less than 12 millimeters;
wherein the attachment region includes one or more engagement members that are configured to shift between an unsecured position and a secured position where the engagement members are secured to the connection region; and
an actuator coupled to the attachment assembly for shifting the one or more engagement members between the unsecured position and the secured position.

2. The connector assembly of claim 1, wherein the plate includes an alignment opening, the alignment opening being configured to allow a sterile barrier adapter to be aligned with the plate.

3. The connector assembly of claim 1, further comprising:
a region of a flexible sterile barrier wedged between the connection region and the attachment assembly, wherein the region lacks an adapter.

4. The connector assembly of claim 1, wherein the one or more engagement members includes one or more wedge tabs configured to engage the circumferential groove.

5. The connector assembly of claim 1, wherein the actuator includes a latch.

6. The connector assembly of claim 1, wherein the plate includes an alignment region.

7. The connector assembly of claim 6, wherein the end effector has an alignment member configured to engage the alignment region of the plate.

8. The connector assembly of claim 1, wherein the securing distance is defined by the depth of the puck.

9. The connector assembly of claim 1, wherein the connection region comprises one or more magnetic field sensors configured to detect one or more magnets of the attachment assembly.

10. A medical assembly, comprising:
a plate configured to be coupled to a robotic arm, the plate having a connection puck and one or more locating regions;
an end effector having an attachment region configured to receive the connection puck and be detachably secured to the connection puck, wherein the attachment region includes:
one or more locating members configured to engage the one or more locating regions of the connection puck so that a sterile barrier can be disposed between the attachment region and the connection puck without requiring a sterile barrier adapter;
one or more engagement members that are configured to shift between an unsecured position and a secured position where the engagement members are secured to the connection puck; and
an actuator coupled to the end effector configured to shift the one or more engagement members between the first position and the secured position.

11. The medical assembly of claim 10, wherein the connection puck has a circumferential groove formed therein.

12. The medical assembly of claim 11, wherein the one or more engagement members includes one or more wedge tabs configured to engage the circumferential groove.

13. The medical assembly of claim 10, wherein the actuator includes a latch.

14. The medical assembly of claim 10, wherein the attachment region is configured to be detachably secured to the connection puck with a securing distance of 12 millimeters or less.

15. The medical assembly of claim 14, wherein the securing distance has a length of 10-12 millimeters.

16. The medical assembly of claim 10, wherein the end effector is a surgical tool guide.

17. A method, comprising:
disposing a region of a sterile barrier lacking an adapter over a connection puck of a plate coupled to a robotic arm, wherein the connection puck includes a plurality of locating regions;
engaging an attachment region of a medical end effector with the connection puck, wherein the engaging includes:
inserting the connection puck into the attachment region, engaging a plurality of locating members of the attachment region with the plurality of locating regions of the connection puck, and
sandwiching the sterile barrier between the attachment region and the connection puck; and
actuating an actuator of the attachment region to shift one or more engagement members from an unsecured position to the secured position.

18. The method of claim 17, wherein actuating the actuator causes one or more wedge tabs of the one or more engagement members to move into a circumferential groove of the connection puck.

19. The method of claim 17, further comprising:
actuating the actuator of the attachment region to shift the one or more engagement members from the secured position and to an unsecured position;
freeing the end effector from the connection puck by axially withdrawing the end effector from the connection puck no more than 12 millimeters; and
moving the end effector laterally after freeing the end effector.

20. The method of claim 17, further comprising:
after actuating the actuator, performing a spinal fusion procedure with the robot arm, the end effector, or both.
